SEQUENCE LISTING (6 sequences)

SEQ ID NO: 1 - RNA, 14 nt, Artificial Sequence
  Synthetic polynucleotide
accacuuccuuucg

SEQ ID NO: 2 - RNA, 10 nt, Artificial Sequence
  Synthetic polynucleotide
acauacaaag

SEQ ID NO: 3 - RNA, 18 nt, Artificial Sequence
  Synthetic polynucleotide
auauuacuucacacaaug

SEQ ID NO: 4 - RNA, 12 nt, Artificial Sequence
  Synthetic polynucleotide
gccuccccccag

SEQ ID NO: 5 - RNA, 24 nt, Artificial Sequence
  Synthetic polynucleotide
ccccuccuccccuuccugcacccg

SEQ ID NO: 6 - RNA, 141 nt, Artificial Sequence
  Synthetic polynucleotide
caaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2018/0237849 A1
(43) Pub. Date: Aug. 23, 2018

(54) RNA MAPPING/FINGERPRINTING

(71) Applicant: ModernaTX, Inc., Cambridge, MA (US)

(72) Inventor: James Thompson, Cambridge, MA (US)

(73) Assignee: ModernaTX, Inc., Cambridge, MA (US)

(21) Appl. No.: 15/753,297

(22) PCT Filed: Aug. 17, 2016
(86) PCT No.: PCT/US16/47416

§ 371 Date: Feb. 17, 2018

Related U.S. Application Data
(60) Provisional application No. 62/206,130, filed on Aug. 17, 2015.

(51) Int. Cl. C12Q 1/6872; C12Q 1/6806

(52) U.S. Cl. CPC C12Q 1/6872; C12Q 2600/158; C12Q 1/6806

(57) ABSTRACT

Novel methods for identification and analysis of mRNA are provided herein. The methods may involve digestion and fingerprinting analysis.

### RELATED APPLICATIONS

This application claims the benefit under 35 U.S.C. 119(e) of U.S. provisional application Ser. No. 62/206,130, filed Aug. 17, 2015, entitled “RNA MAPPING/FINGERPRINTING”, the entire contents of which are incorporated by reference herein.

### FIELD OF THE INVENTION

The present disclosure relates generally to the field of biotechnology and more specifically to the field of analytical chemistry.

### BACKGROUND OF THE INVENTION

It is of great interest in the fields of therapeutics, diagnostics, reagents and for biological assays to be able to design, synthesize and deliver a nucleic acid, e.g., a ribonucleic acid (RNA) for example, a messenger RNA (mRNA) inside a cell, whether in vitro, in vivo, in situ or ex vivo, such as to effect physiologic outcomes which are beneficial to the cell, tissue or organ and ultimately to an organism. One beneficial outcome is to cause intracellular translation of the nucleic acid and production of at least one encoded peptide or polypeptide of interest. In some cases, RNA is synthesized in the laboratory in order to achieve these methods.

### SUMMARY OF THE INVENTION

The validation and/or purification of synthesized RNA is important, particularly in therapeutic methods. Novel methods of identifying mRNA molecules are provided. In some aspects, methods described by the disclosure are useful for validating the production of therapeutic mRNA molecules. For example, laboratory-synthesized (e.g., by in vitro transcription) mRNA molecules encoding a protein of therapeutic relevance should be analyzed to ensure the absence of product-related impurities (e.g., less than full-length mRNAs, degradants, or read-through transcripts that are longer than the intended mRNA product), process-related impurities (e.g., nucleic acids and/or reagents carried over from synthesis reactions), or contaminants (e.g., exogenous or adventitious nucleic acids) from the mRNA molecules prior to administration to a subject.

In some aspects the invention is a method for determining the presence of an RNA in a mRNA sample, by determining a signature profile of the mRNA sample, comparing the signature profile to a known signature profile for a test mRNA, identifying the presence of an RNA in the mRNA sample based on a comparison with the known signature profile for the test mRNA. In other aspects the invention is a method for determining the presence of an RNA in a mRNA sample, by determining a signature profile of the mRNA sample, comparing the profile of the masses of the fragments generated to the predicted masses from the primary molecular sequence of the mRNA (e.g., a theoretical pattern), identifying the presence of an RNA in the mRNA sample based on the theoretical versus observed mass pattern. In some embodiments the RNA is an impurity in the mRNA sample if the signature profile of the mRNA sample does not match the known signature profile for the test mRNA. In other embodiments the method has a sensitivity threshold such that an impurity of less than 1% of the sample is detected.

In other embodiments the method further involves identifying the presence of the test mRNA if the known signature profile for the test mRNA is included within the signature profile of the mRNA sample. In some embodiments the signature profile of the mRNA sample is determined by a method that includes a digestion step and a separation/detection step.

Accordingly, in other aspects the disclosure provides a method for confirming the identity of a test mRNA, the method comprising: (a) digesting a test mRNA with enzyme nuclease (e.g., an endonuclease, such as an RNase enzyme) to produce a plurality of mRNA fragments; (b) physically separating the plurality of mRNA fragments; (c) assigning a signature to the test mRNA by detecting the plurality of fragments; (d) identifying the test mRNA by comparing the signature to a known mRNA signature, and (e) confirming the identity of the test mRNA if the signature of the test mRNA is the same as the known mRNA signature.

In other aspects the disclosure provides a method for confirming the identity of a test mRNA, the method comprising: (a) digesting a test mRNA with an RNase enzyme to produce a plurality of mRNA fragments; (b) physically separating the plurality of mRNA fragments; (c) determining the masses of the fragments; (d) identifying the test mRNA by comparing the signature to the predicted mass pattern (e.g., a theoretical pattern), and (e) confirming the identity of the test mRNA if the observed masses match theoretical.

In some embodiments, the target mRNA is an in vitro transcribed RNA (IVT mRNA). In some embodiments, the target mRNA is a therapeutic mRNA. In some embodiments, the RNase enzyme is RNase T1.

In some embodiments, the digesting occurs in a buffer. In some embodiments, the buffer comprises at least one component selected from the group consisting of: urea, EDTA, magnesium chloride (MgCl₂) and Tris. In some embodiments, the buffer further comprises 2′,3′-Cyclic-nucleotide 3′-phosphodiesterase (CNP) and/or Calf Intestinal Alkaline Phosphatase (CIP). In some embodiments, the digestion occurs at about 37° C.

In some embodiments, the physical separation and/or the detecting is achieved by a method selected from the group consisting of: gel electrophoresis, high pressure liquid chromatography (HPLC), and mass spectrometry. In some embodiments, the HPLC is HPLC-UV. In some embodiments, the mass spectrometry is Electrospray Ionization mass spectrometry (ESI-MS) or Matrix-assisted Laser Desorption/Ionization-Time of Flight (MALDI-TOF) mass spectrometry.

In some embodiments, the signature assigned to the test mRNA is an absorbance spectrum or a mass spectrum.

In some embodiments, the signature of the test mRNA shares at least 70%, at least 80%, at least 90%, at least 95%, at least 99%, or at least 99.9% identity with the known mRNA signature.

In some embodiments, the test mRNA is removed from a population of mRNAs that will be administered as a therapeutic to a subject in need thereof.

A method for quality control of an RNA pharmaceutical composition is provided according to other aspects of the invention. The method involves digesting the RNA pharmaceutical composition with an RNase enzyme to produce a plurality of RNA fragments; physically separating the plurality of RNA fragments; generating a signature profile of the RNA pharmaceutical composition by detecting the plurality of fragments; comparing the signature profile with a known RNA signature profile, and determining the quality of the RNA based on the comparison of the signature profile with the known RNA signature profile. In some embodiments, the signature profile of the mRNA sample, is compared to the predicted masses from the primary molecular sequence of the mRNA (e.g., a theoretical pattern).

A pure mRNA sample, having a composition of an in vitro transcribed (IVT) RNA and a pharmaceutically acceptable carrier, that is preparable according to any of the methods described herein is provided in other aspects of the invention.

In other aspects of the invention a system for determining batch purity of an RNA pharmaceutical composition comprising: a computing system; at least one electronic database coupled to the computing system; at least one software routine executing on the computing system which is programmed to: (a) receive data comprising an RNA fingerprint of the RNA pharmaceutical composition; (b) analyze the data; (c) based on the analyzed data, determine batch purity of the RNA pharmaceutical composition is provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

FIG. 1 shows the total number of RNA fragments predicted to be generated by RNase T1 digestion of mRNA Sample 1. For example, there are 92 2-mer fragments generated by this digestion.

FIG. 2 shows the number of unique fragments predicted to be generated by RNase T1 digestion of mRNA Sample 1. For example, there are 31 unique 6-mer fragments generated by this RNase digestion.

FIG. 3 shows the mass of different fragment lengths predicted to be generated. For example, 10% of the total mass of mRNA sample 1 is digested into 6-mers.

FIG. 4 shows analyses of Sample 1 after RNase T1 digestion by HPLC produces a chromatographic pattern that represents a unique fingerprint for Sample 1.

FIG. 5 shows representative HPLC data demonstrating the reproducibility of RNase digestion. Two samples of mRNA Sample 1 were digested and run on an HPLC column. The trace patterns for each digestion of mRNA Sample 1 (e.g., Run 1 and Run 2) demonstrate good peak alignments.

FIG. 6 shows representative HPLC data demonstrating the unique pattern generated by RNase digestion of two different mRNA samples (e.g., mRNA Sample 1 and mRNA Sample 2) demonstrating poor peak alignments, thereby enabling differentiation of these two samples.

FIG. 7 shows representative HPLC data demonstrating the reproducibility of RNase digestion across multiple digests. Separate aliquots of mRNA Sample 3 were RNase digested (Digest 1, 2 and 3) and run on an HPLC column. The trace patterns for each digestion demonstrate good peak alignments.

FIG. 8 shows representative HPLC data illustrating that digestion with different RNase enzymes (e.g., RNase T1 or RNase A) leads to the generation of distinct trace patterns. Digestion of mRNA Sample 3 with RNase T1 provides a trace pattern exhibiting greater complexity than digestion with RNase A.

FIG. 9 shows representative ESI-MS data. Two mRNA samples (mRNA Sample 1 and mRNA Sample 2) were digested with RNase T1. ESI-MS was performed on digested samples. Results demonstrate that unique mass traces are generated for each sample.

FIGS. 10A-10B show representative data from ESI-MS of two RNase T1-digested mRNA samples (mRNA Sample 4 and mRNA Sample 5). Data demonstrates that each mass fingerprint is unique.

FIG. 11 shows representative data from LC/MS of RNase T1-digested mRNA encoding mCherry.

### DETAILED DESCRIPTION OF THE INVENTION

Delivery of mRNA molecules to a subject in a therapeutic context is promising because it enables intracellular translation of the mRNA and production of at least one encoded peptide or polypeptide of interest without the need for nucleic acid-based delivery systems (e.g., viral vectors and DNA-based plasmids). Therapeutic mRNA molecules are generally synthesized in a laboratory (e.g., by in vitro transcription). However, there is a potential risk of carrying over impurities or contaminants, such as incorrectly synthesized mRNA and/or undesirable synthesis reagents, into the final therapeutic preparation during the production process. In order to prevent the administration of impure or contaminated mRNA, the mRNA molecules can be subject to a quality control (QC) procedure (e.g., validated or identified) prior to use. Validation confirms that the correct mRNA molecule has been synthesized and is pure.

Typical assays for examining the purity of an RNA sample do not achieve the level of accuracy that can be achieved by the direct structural characterization involving RNA fingerprinting of the instant methods. According to some aspects of the invention a method of analyzing and characterizing an RNA sample is provided. The method involves determining a signature profile of the mRNA sample, comparing the signature profile to a known signature profile for a test mRNA, identifying the presence of an RNA in the mRNA sample based on a comparison with the known signature profile for the test mRNA.

In other aspects the invention is a method for determining the presence of an RNA in a mRNA sample, by determining a signature profile of the mRNA sample, comparing the profile of the masses of the fragments generated to the predicted masses from the primary molecular sequence of the RNA (e.g., a theoretical pattern), identifying the presence of an RNA in the mRNA sample based on the theoretical versus observed mass pattern.

The methods of the invention can be used for a variety of purposes where the ability to identify and RNA fingerprint is important. For instance, the methods of the invention are useful for monitoring batch-to-batch variability of an RNA composition or sample. The purity of each batch may be determined by determining any differences in the signature profile in comparison to a known signature profile or a theoretical profile of predicted masses from the primary molecular sequence of the RNA. These signatures are also useful for monitoring the presence of unwanted nucleic acids which may be active components in the sample. The methods may also be performed on at least two samples to determine which sample has better purity or to otherwise compare the purity of the samples.

Thus, in some instances the methods of the invention are used to determine the purity of an RNA sample. The term “pure” as used herein refers to material that has only the target nucleic acid active agents such that the presence of unrelated nucleic acids is reduced or eliminated, i.e., impurities or contaminants, including RNA fragments. For example, a purified RNA sample includes one or more target or test nucleic acids but is preferably substantially free of other nucleic acids. As used herein, the term “substantially free” is used operationally, in the context of analytical testing of the material. Preferably, purified material substantially free of impurities or contaminants is at least 95% pure; more preferably, at least 98% pure, and more preferably still at least 99% pure. In some embodiments a pure RNA sample is comprised of 100% of the target or test RNAs and includes no other RNA. In some embodiments it only includes a single type of target or test RNA.

A “polynucleotide” or “nucleic acid” is at least two nucleotides covalently linked together, and in some instances, may contain phosphodiester bonds (e.g., a phosphodiester “backbone”) or modified bonds, such as phosphorothioate bonds. An “engineered nucleic acid” is a nucleic acid that does not occur in nature. In some instances the RNA in the RNA sample is an engineered RNA sample. It should be understood, however, that while an engineered nucleic acid as a whole is not naturally-occurring, it may include nucleotide sequences that occur in nature. Thus, a “polynucleotide” or “nucleic acid” sequence is a series of nucleotide bases (also called “nucleotides”), generally in DNA and RNA, and means any chain of two or more nucleotides. The terms include genomic DNA, cDNA, RNA, any synthetic and genetically manipulated polynucleotide. This includes single- and double-stranded molecules; i.e., DNA-DNA, DNA-RNA, and RNA-RNA hybrids as well as “protein nucleic acids” (PNA) formed by conjugating bases to an amino acid backbone.

The methods of the invention involve the analysis of RNA samples. An RNA in an RNA sample typically is composed of repeating ribonucleosides. It is possible that the RNA includes one or more deoxyribonucleosides. In preferred embodiments the RNA is comprised of greater than 60%, 70%, 80% or 90% of ribonucleosides. In other embodiments the RNA is 100% comprised of ribonucleosides. The RNA in an RNA sample is preferably an mRNA.

As used herein, the term “messenger RNA (mRNA)” refers to a ribonucleic acid that has been transcribed from a DNA sequence by an RNA polymerase enzyme, and interacts with a ribosome to synthesize protein encoded by DNA. Generally, mRNA are classified into two sub-classes: pre-mRNA and mature mRNA. Precursor mRNA (pre-mRNA) is mRNA that has been transcribed by RNA polymerase but has not undergone any post-transcriptional processing (e.g., 5′capping, splicing, editing, and polyadenylation). Mature mRNA has been modified via post-transcriptional processing (e.g., spliced to remove introns and polyadenylated region) and is capable of interacting with ribosomes to perform protein synthesis.

mRNA can be isolated from tissues or cells by a variety of methods. For example, a total RNA extraction can be performed on cells or a cell lysate and the resulting extracted total RNA can be purified (e.g., on a column comprising oligo-dT beads) to obtain extracted mRNA.

Alternatively, mRNA can be synthesized in a cell-free environment, for example by in vitro transcription (IVT). IVT is a process that permits template-directed synthesis of ribonucleic acid (RNA) (e.g., messenger RNA (mRNA)). It is based, generally, on the engineering of a template that includes a bacteriophage promoter sequence upstream of the sequence of interest, followed by transcription using a corresponding RNA polymerase. In vitro mRNA transcripts, for example, may be used as therapeutics in vivo to direct ribosomes to express protein therapeutics within targeted tissues.

Traditionally, the basic components of an mRNA molecule include at least a coding region, a 5′UTR, a 3′UTR, a 5′ cap and a poly-A tail. IVT mRNA may function as mRNA but are distinguished from wild-type mRNA in their functional and/or structural design features which serve to overcome existing problems of effective polypeptide production using nucleic-acid based therapeutics. For example, IVT mRNA may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

An RNA may comprise naturally occurring nucleotides and/or non-naturally occurring nucleotides such as modified nucleotides. In some embodiments, the RNA polynucleotide of the RNA vaccine includes at least one chemical modification. In some embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. Other exemplary chemical modifications useful in the mRNA described herein include those listed in US Published patent application 2015/0064235.

In some embodiments the methods may be used to detect differences in chemical modification of an mRNA sample. The presence of different chemical modifications patterns may be detected using the methods described herein.

An “in vitro transcription template (IVT),” as used herein, refers to deoxyribonucleic acid (DNA) suitable for use in an IVT reaction for the production of messenger RNA (mRNA). In some embodiments, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template.

A “5′ untranslated region (UTR)” refers to a region of an mRNA that is directly upstream (i.e., 5′) from the start codon (i.e., the first codon of an mRNA transcript translated by a ribosome) that does not encode a protein or peptide.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

An “open reading frame” is a continuous stretch of DNA beginning with a start codon (e.g., methionine (ATG)), and ending with a stop codon (e.g., TAA, TAG or TGA) and encodes a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation.

In some embodiments, the test or target mRNA (e.g., IVT mRNA) is a therapeutic mRNA. As used herein, the term “therapeutic mRNA” refers to an mRNA molecule (e.g., an IVT mRNA) that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders.

A “test mRNA” or “target mRNA” (used interchangeably herein) is an mRNA of interest, having a known nucleic acid sequence. The test mRNA may be found in a RNA or mRNA sample. In addition to the test mRNA the RNA or mRNA sample may include a plurality of mRNA molecules or other impurities obtained from a larger population of mRNA molecules. For example, after the production of IVT mRNA, a test mRNA sample may be removed from the population of IVT mRNA in order to assay for the purity and/or to confirm the identity of the mRNA produced by IVT.

In some embodiments, the test mRNA is assigned a signature, referred to as a signature profile for a test mRNA. As used herein, the term “signature” refers to a unique identifier or fingerprint that uniquely identifies an mRNA. A “signature profile for a test mRNA” is a signature generated from an mRNA sample suspected of having a test mRNA based on fragments generated by digestion with a particular RNase enzyme. For example, digestion of an mRNA with RNase T1 and subsequent analysis of the resulting plurality of mRNA fragments by HPLC or mass spec produces a trace or mass profile, or signature that can only be created by digestion of that particular mRNA with RNase T1.

In other embodiments, test mRNA is digested with RNaseH. RNaseH cleaves the 3′-O—P bond of RNA in a DNA/RNA duplex substrate to produce 3′-hydroxyl and 5′-phosphate terminated products. Therefore, specific DNA oligos can be designed to anneal to the test mRNA, and the resulting duplexes digested with RNase H to generate a unique fragment pattern (resulting in a unique mass profile) for a given test mRNA.

Once the signature of a mRNA sample is determined it can be compared with a known signature profile for a test mRNA. A “known signature profile for a test mRNA” as used herein refers to a control signature or fingerprint that uniquely identifies the test mRNA. The known signature profile for a test mRNA may be generated based on digestion of a pure sample and compared to the test signature profile. Alternatively it may be a known control signature, stored in a electronic or non-electronic data medium. For example, a control signature may be a theoretical signature based on predicted masses from the primary molecular sequence of a particular RNA (e.g., a test mRNA).

Various batches of mRNA (e.g., test mRNA) can be digested under the same conditions and compared to the signature of the pure mRNA to identify impurities or contaminants (e.g., additives, such as chemicals carried over from IVT reactions, or incorrectly transcribed mRNA) or to a known signature profile for the test mRNA. The identity of a test mRNA may be confirmed if the signature of the test mRNA shares identity with the known signature profile for a test mRNA. In some embodiments, the signature of the test mRNA shares at least 60%, at least 65%, at least 70%, at least 80%, at least 90%, at least 95%, at least 99%, or at least 99.9% identity with the known mRNA signature.

In some embodiments, various batches of mRNA can be digested under the same conditions in a high throughput fashion. For example, each mRNA sample of a batch may be placed in a separate well or wells of a multi-well plate and digested simultaneously with an RNase. A multi-well plate can comprise an array of 6, 24, 96, 384 or 1536 wells. However, the skilled artisan recognizes that multi-well plates may be constructed into a variety of other acceptable configurations, such as a multi-well plate having a number of wells that is a multiple of 6, 24, 96, 384 or 1536. For example, in some embodiments, the multi-well plate comprises an array of 3072 wells (which is a multiple of 1536). The number of mRNA samples digested simultaneously (e.g., in a multi-well plate) can vary. In some embodiments, at least two mRNA samples are digested simultaneously. In some embodiments, between 2 and 96 mRNA samples are digested simultaneously. In some embodiments, between 2 and 384 mRNA samples are digested simultaneously. In some embodiments, between 2 and 1536 mRNA samples are digested simultaneously. The skilled artisan recognizes that mRNA samples being digested simultaneously can each encode the same protein, or different proteins (e.g., mRNA encoding variants of the same protein, or encoding a completely different protein, such as a control mRNA).

As used herein, the term “digestion” refers to the enzymatic degradation of a biological macromolecule. Biological macromolecules can be proteins, polypeptides, or nucleic acids (e.g., DNA, RNA, mRNA), or any combination of the foregoing. Generally, the enzyme that mediates digestion is a protease or a nuclease, depending upon the substrate on which the enzyme performs its function. Proteases hydrolyze the peptide bonds that link amino acids in a peptide chain. Examples of proteases include but are not limited to serine proteases, threonine proteases, cysteine proteases, aspartase proteases, and metalloproteases. Nucleases cleave phosphodiester bonds between nucleotide subunits of nucleic acids. Generally, nucleases can be classified as deoxyribonucleases, or DNase enzymes (e.g., nucleases that cleave DNA), and ribonucleases, or RNase enzymes (e.g., nucleases that cleave RNA). Examples of DNase enzymes include exodeoxyribonucleases, which cleave the ends of DNA molecules, and restriction enzymes, which cleave specific sequences with a DNA sequence.

The amount of test mRNA that is digested can vary. In some embodiments that amount of test mRNA that is digested ranges from about 1 ng to about 100 μg. In some embodiments, the amount of test mRNA that is digested ranges from about 10 ng to about 80 μg. In some embodiments, the amount of test mRNA that is digested ranges from about 100 ng to about 1000 μg. In some embodiments, the amount of test mRNA that is digested ranges from about 500 ng to about 40 μg. In some embodiments, the amount of test mRNA that is digested ranges from about 1 μg to about 35 μg. In some embodiments, the amount of mRNA that is digested is about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, or about 30 μg.

The disclosure relates, in part, to the discovery that RNase enzymes can be used to digest mRNA to create a unique population of RNA fragments, or a “signature”. Examples of RNase enzymes include but are not limited to RNase A, RNaseH, RNase III, RNase L, RNase P, RNase E, RNase PhyM, RNase T1, RNase T2, RNase U2, RNase V, RNase PH, RNase R, RNase D, RNase T, polynucleotide phosphorylase (PNPase), oligoribonuclease, exoribonuclease I, and exoribonuclease II. In some embodiments, RNase T1 or RNase A is used to determine the identity of a test mRNA.

The concentration of RNase enzyme used in methods described by the disclosure can vary depending upon the amount of mRNA to be digested. However, in some embodiments, the amount of RNase enzyme ranges between about 0.1 Unit and about 500 Units of RNase. In some embodiments, the amount of RNase enzyme ranges from about 0.1 U to about 1 U, 1 U to about 5 U, 2 U to about 200 U, 10 U to about 450 U, about 20 U to about 400 U, about 30 U to about 350 U, about 40 U to about 300 U, about 50 U to about 250 U, or about 100 U to about 200 U. In some embodiments, the amount of RNase enzyme ranges between about 500 Units to about 3000 Units of RNase (e.g., about 500, 1000, 1500, 2000, 2500, or 3000 Units of RNase).

The skilled artisan also recognizes that RNase enzymes can be derived from a variety of organisms, including but not limited to animals (e.g., mammals, humans, cats, dogs, cows, horses, etc.), bacteria (e.g., E. coli, S. aureus, Clostridium spp., etc.), and mold (e.g., Aspergillus oryzae, Aspergillus niger, Dictyostelium discoideum, etc.). RNase enzymes may also be recombinantly produced. For example, a gene encoding an RNase enzyme from one species (e.g., RNase T1 from A. oryzae) can be heterologously expressed in a bacterial host cell (e.g., E. coli) and purified. In some embodiments, the digestion is performed by an A. oryzae RNase T1 enzyme.

In some embodiments, the digestion is performed in a buffer. As used herein, the term “buffer” refers to a solution that can neutralize either an acid or a base in order to maintain a stable pH. Examples of buffers include but are not limited to Tris buffer (e.g., Tris-Cl buffer, Tris-acetate buffer, Tris-base buffer), urea buffer, bicarbonate buffer (e.g., sodium bicarbonate buffer), HEPES (4-2-hydroxyethyl-1-piperazineethanesulfonic acid) buffer, MOPS (3-(N-morpholino)propanesulfonic acid) buffer, PIPES (piperazine-N,N′-bis(2-ethanesulfonic acid)) buffer, and Triethylammonium acetate (TEAAc buffer). A buffer can also contain more than one buffering agent, for example Tris-Cl and urea. The concentration of each buffering agent in a buffer can range from about 1 mM to about 10 M. In some embodiments, the concentration of each buffering agent in a buffer ranges from about 1 mM to about 20 mM, about 10 mM to about 50 mM, about 25 mM to about 100 mM, about 75 mM to about 200 mM, about 100 mM to about 500 mM, about 250 mM to about 1 M, about 500 mM to about 3 M, about 1 M to about 5 M, about 3 M to about 8 M, or about 5 M to about 10 M.

Generally, the pH maintained by a buffer can range from about pH 6.0 to about pH 10.0. In some embodiments, the pH can range from about pH 6.8 to about 7.5. In some embodiments, the pH is about pH 6.5, about pH 6.6, about pH 6.7, about pH 6.8, about pH 6.9, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, or about pH 10.

In some embodiments, a buffer further comprises a chelating agent. Examples of chelating agents include, but are not limited to, ethylenediaminetetraacetic acid (EDTA), ethylene glycol tetra acetic acid (EGTA), dimercapto succinic acid (DMSA), and 2,3-dimercapto-1-propanesulfonic acid (DMPS). In some embodiments, the chelating agent is EDTA (ethylenediaminetetraacetic acid). The concentration of EDTA can range from about 1 mM to about 500 mM. In some embodiments, the concentration of EDTA ranges from about 10 mM to about 300 mM. In some embodiments, the concentration of EDTA ranges from about 20 mM to about 250 mM EDTA.

The skilled artisan recognizes that to facilitate digestion, mRNA can be denatured prior to incubation with an RNase enzyme. In some embodiments, mRNA is denatured at a temperature that is at least 50° C., at least 60° C., at least 70° C., at least 80° C., or at least 90° C. Digestion of a test mRNA can be carried out at any temperature at which the RNase enzyme will perform its intended function. The temperature of a test mRNA digestion reaction can range from about 20° C. to about 100° C. In some embodiments, the temperature of a test mRNA digestion reaction ranges from about 30° C. to about 50° C. In some embodiments, a test mRNA is digested by an RNase enzyme at 37° C.

Digestion with RNase enzymes may lead to the formation of cyclic phosphates and other intermediates (e.g., 2′ or 3′-phosphates) that can interfere with downstream processing (e.g., detection of digested test mRNA fragments). Thus, in some embodiments, an mRNA digestion buffer further comprises agents that disrupt or prevent the formation of intermediates. In some embodiments, the buffer further comprises 2′,3′-Cyclic-nucleotide 3′-phosphodiesterase (CNP) and/or Alkaline Phosphatase, such as Calf Intestinal Alkaline Phosphatase (CIP), or Shrimp Alkaline Phosphatase (SAP). The concentration of each agent that disrupts or prevents formation of intermediates can range from about 10 ng/μL to about 100 ng/μL. In some embodiments, the concentration of each agent ranges from about 15 ng/μL to about 25 ng/μL. Alternatively, or in combination with the above-stated concentration range, the amount of agent can range from about 1 U to about 50 U, about 2 U to about 40 U, about 3 U to about 35 U, about 4 U to about 30 U, about 5 U to about 25 U, or about 10 U to about 20 U. In some embodiments, digestion with RNase enzymes is performed in a digestion buffer not containing CIP and/or CNP.

In some embodiments, a buffer further comprises magnesium chloride (MgCl₂). Generally, MgCl₂ can act as a cofactor for enzyme (e.g., RNase) activity. The concentration of MgCl₂ in the buffer ranges from about 0.5 mM to about 200 mM. In some embodiments, the concentration of MgCl₂ in the buffer ranges from about 0.5 mM to about 10 mM, 1 mM to about 20 mM, 5 mM to about 20 mM, 10 mM to about 75 mM, or about 50 mM to about 150 mM. In some embodiments, the concentration of MgCl₂ in the buffer is about 1 mM, about 5 mM, about 10 mM, about 50 mM, about 75 mM, about 100 mM, about 125 mM, or about 150 mM.

In some embodiments, digestion of a test mRNA comprises two incubation steps: (a) RNase digestion of test mRNA, and (b) processing of digested test mRNA. In some embodiments, digestion of a test mRNA further comprises the step of denaturing test mRNA prior to digestion. The incubation time for each of the above steps (a), (b), and (c) can range from about 1 minute to about 24 hours. In some embodiments, incubation time ranges from about 1 minute to about 10 minutes (e.g., about 1, 2, 3, 4, 5, 6, 7, 8, 9, or 10 minutes). In some embodiments, incubation time ranges from about 5 minutes to about 15 minutes (e.g. about 5, 6, 7, 8, 9, 10, 11, 12, 13, 14, or 15 minutes). In some embodiments, incubation time ranges from about 30 minutes to about 4 hours (240 minutes). In some embodiments, incubation time ranges from about 1 hour to about 5 hours. In some embodiments, incubation time ranges from about 2 hours to about 12 hours. In some embodiments, incubation time ranges from about 6 hours to about 24 hours.

The skilled artisan recognizes that digestions may be carried out under various environmental conditions based upon the components present in the digestion reaction. Any suitable combination of the foregoing components and parameters may be used. For example, digestion of a test mRNA may be carried out according to the protocol set forth in Table 1.

A “fragment” of a polynucleotide of interest comprises a series of consecutive nucleotides from the sequence of said test RNA. By way of example, a “fragment” of a polynucleotide of interest may comprise (or consist of) at least 1 at least 2, at least 5, at least 10, at least 20, at least 30 consecutive nucleotides from the sequence of the polynucleotide (e.g., at least 1 at least 2, at least 5, at least 10, at least 20, at least 30, at least 35, 50, 75, 100, 150, 200, 250, 300, 350, 400, 450, 500, 550, 600, 650, 700, 750, 800, 850, 900, 950 or 1000 consecutive nucleic acid residues of said polynucleotide). A fragment of a polynucleotide (e.g., an mRNA fragment) can consist of the same nucleotide sequence as another fragment, or consist of a unique nucleotide sequence.

A “plurality of mRNA fragments” refers to a population of at least two mRNA fragments. mRNA fragments comprising the plurality can be identical, unique, or a combination of identical and unique (e.g., some fragments are the same and some are unique). The skilled artisan recognizes that fragments can also have the same length but comprise different nucleotide sequences (e.g., CACGU, and AAAGC are both five nucleotides in length but comprise different sequences). In some embodiments, a plurality of mRNA fragments is generated from the digestion of a single species of mRNA. A plurality of mRNA fragments can be at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, or at least 500 mRNA fragments. In some embodiments, a plurality of mRNA fragments comprises more than 500 mRNA fragments.

The plurality of fragments is physically separated. As used herein, the term “physically separated” refers to the isolation of mRNA fragments based upon a selection criteria. For example, a plurality of mRNA fragments resulting from the digestion of a test mRNA can be physically separated by chromatography or mass spectrometry. In some embodiments, fragments of a test mRNA can be physically separated by capillary electrophoresis to generate an electropherogram. Examples of chromatography methods include size exclusion chromatography and high performance liquid chromatography (HPLC). Examples of mass spectrometry physical separation techniques include electrospray ionization mass spectrometry (ESI-MS) and matrix-assisted laser desorption ionization time of flight (MALDI-TOF). In some embodiments, each of fragment of the plurality of mRNA fragments is detected during the physical separation. For example, a UV spectrophotometer coupled to a HPLC machine can be used to detect the mRNA fragments during physical separation (e.g., an absorbance spectrum profile). The resulting data, also called a “trace” provides a graphical representation of the composition of the plurality of mRNA fragments. In another embodiment, a mass spectrophotometer generates mass data during the physical separation of a plurality of mRNA fragments. The graphic depiction of the mass data can provide a “mass fingerprint” that identifies the contents of the plurality of mRNA fragments.

Mass spectrometry encompasses a broad range of techniques for identifying and characterizing compounds in mixtures. Different types of mass spectrometry-based approaches may be used to analyze a sample to determine its composition. Mass spectrometry analysis involves converting a sample being analyzed into multiple ions by an ionization process. Each of the resulting ions, when placed in a force field, moves in the field along a trajectory such that its acceleration is inversely proportional to its mass-to-charge ratio. A mass spectrum of a molecule is thus produced that displays a plot of relative abundances of precursor ions versus their mass-to-charge ratios. When a subsequent stage of mass spectrometry, such as tandem mass spectrometry, is used to further analyze the sample by subjecting precursor ions to higher energy, each precursor ion may undergo disassociation into fragments referred to as product ions. Resulting fragments can be used to provide information concerning the nature and the structure of their precursor molecule.

MALDI-TOF (matrix-assisted laser desorption ionization time of flight) mass spectrometry provides for the spectrometric determination of the mass of poorly ionizing or easily-fragmented analytes of low volatility by embedding them in a matrix of light-absorbing material and measuring the weight of the molecule as it is ionized and caused to fly by volatilization. Combinations of electric and magnetic fields are applied on the sample to cause the ionized material to move depending on the individual mass and charge of the molecule. U.S. Pat. No. 6,043,031, issued to Koster et al., describes an exemplary method for identifying single-base mutations within DNA using MALDI-TOF and other methods of mass spectrometry.

HPLC (high performance liquid chromatography) is used for the analytical separation of bio-polymers, based on properties of the bio-polymers. HPLC can be used to separate nucleic acid sequences based on size and/or charge. A nucleic acid sequence having one base pair difference from another nucleic acid can be separated using HPLC. Thus, nucleic acid samples, which are identical except for a single nucleotide may be differentially separated using HPLC, to identify the presence or absence of a particular nucleic acid fragments. Preferably the HPLC is HPLC-UV.

The data generated using the methods of the invention can be processed individually or by a computer. For instance, a computer-implemented method for generating a data structure, tangibly embodied in a computer-readable medium, representing a data set representative of a signature profile of an RNA sample may be performed according to the invention.

Some embodiments relate to at least one non-transitory computer-readable storage medium storing computer-executable instructions that, when executed by at least one processor, perform a method of identifying an RNA in a sample.

Thus, some embodiments provide techniques for processing MS/MS data that may identify impurities in a sample with improved accuracy, sensitivity and speed. The techniques may involve structural identification of an RNA fragment regardless of whether it has been previously identified and included in a reference database. A scoring approach may be utilized that allows determining a likelihood of an impurity being present in a sample, with scores being computed so that they do not depend on techniques used to acquire the analyzed mass spectrometry data.

In some embodiments the known signature profile for known mRNA data may be computationally generated, or computed, and stored, for example, in a first database. The first database may store any type of information on the RNA, including an identifier of each RNA fragment to form a complete signature and any other suitable information. In some embodiments, a score may be computed for each set of computed fragments retrieved from a second database including the known signatures, the score indicating correlation between the set of known signatures and the set of experimentally obtained fragments. To compute the score, for example, each fragment in a set of computed fragments matching a corresponding fragment in the set of experimentally obtained fragments may be assigned a weight based on a relative abundance of the experimentally obtained fragment. A score may thus be computed for each set of computed fragments based on weights assigned to fragments in that set. The scores may then be used to identify difference between the RNA sample and the known sequence.

A computer system that may implement the above as a computer program typically may include a main unit connected to both an output device which displays information to a user and an input device which receives input from a user. The main unit generally includes a processor connected to a memory system via an interconnection mechanism. The input device and output device also may be connected to the processor and memory system via the interconnection mechanism.

An illustrative implementation of a computer system that may be used in connection with some embodiments may be used to implement any of the functionality described above. The computer system may include one or more processors and one or more computer-readable storage media (i.e., tangible, non-transitory computer-readable media), e.g., volatile storage and one or more non-volatile storage media, which may be formed of any suitable data storage media. The processor may control writing data to and reading data from the volatile storage and the non-volatile storage device in any suitable manner, as embodiments are not limited in this respect. To perform any of the functionality described herein, the processor may execute one or more instructions stored in one or more computer-readable storage media (e.g., volatile storage and/or non-volatile storage), which may serve as tangible, non-transitory computer-readable media storing instructions for execution by the processor.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation comprises at least one computer-readable storage medium (i.e., at least one tangible, non-transitory computer-readable medium), such as a computer memory (e.g., hard drive, flash memory, processor working memory, etc.), a floppy disk, an optical disk, a magnetic tape, or other tangible, non-transitory computer-readable medium, encoded with a computer program (i.e., a plurality of instructions), which, when executed on one or more processors, performs above-discussed functions. The computer-readable storage medium can be transportable such that the program stored thereon can be loaded onto any computer resource to implement techniques discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-techniques.

### EXAMPLES

### Example 1: RNAase Mapping/Fingerprinting Example Protocol

Table 1 (below) demonstrates an example protocol for RNase digestion:

TABLE 1
Example protocol for RNase T1 digestion.

| RNase T1 Fingerprint with | | | | |
|---|---|---|---|---|
| UREA Buffer | | Concentration | | Source |
| 10.0 μl | mRNA | 3 | mg/ml | |
| 15.0 μl | UREA Solution, | 8000 | mM | UREA Solution 8M, |
| | Sigma | | | Sigma 51457 |
| 3.0 μl | Tris, pH 7 | 1000 | mM | Tris-Cl Buffer, pH 7, |
| | | | | Sigma, T1819 |
| 2.0 μl | EDTA | 50 | mM | EDTA, 0.5M, pH 8, |
| | | | | Applichem, A4892.0500 |
| →10 min @ 90° C. | | | | |
| 20.0 μl | RNase T1 | 10.0 | U/μl | RNase, T1, Thermo, |
| | | | | #EN0542 |
| →3 hr @ 37° C. | | | | |
| 2.0 μl | CNP | 0.040 | μg/μl | CNP, Origene, TP602895 |
| 2.0 μl | MgCI₂ | 100 | mM | MgCI2, 1M, Ambion, |
| | | | | AM9530G |
| →1 h @37° C. | | | | |
| 2.0 μl | CIP | 10.0 | U/μl | CIP, New England |
| | | | | BioLabs, M0290L |

→1 h @ 37° C.

Stop Incubation

| | |
|---|---|
| 5.0 μl | 250 mM EDTA, |
| | 1M TEAAc |
| 61.0 μl | Total Sample |
| | Volume |

Briefly, a mRNA sample was denatured at high temperature in a urea buffer. RNase (e.g., RNAase T1) was added to the denatured sample and incubated. 2′,3′-phosphates were digested for 1 hour with cyclic-nucleotide 3′-phosphodiesterase (CNP) at 37° C. The resultant 2′- or 3′ phosphates were removed by digestion with Calf Intestinal Alkaline Phosphatase (CIP). The digestion was stopped by the addition of EDTA. TEAAc was also added for strong adsorption on the HPLC column. After the reaction was stopped, the digested mRNA sample was prepared for analysis using HPLC. Suitable analysis methods include IP-RP-HPLC, HPLC-UV, AEX-HPLC, ESI-MS and/or MALDI-ToF, some of which are described below.

### Identification of RNA Using RNase Fingerprinting

A first mRNA sample (sample 1) was processed according the methods described above. A table summarizing theoretical RNase T1 cleavage products from that analysis is provided below in Table 2.

TABLE 2
Theoretical RNase T1 cleavage products.

| | # Unique | |
|---|---|---|
| | Fragments | Prevalence |
| 1 mers | 1 | 152 |
| 2 mers | 4 | 92 |
| 3 mers | 9 | 71 |
| 4 mers | 20 | 52 |
| 5 mers | 23 | 29 |
| 6 mers | 31 | 34 |
| 7 mers | 23 | 24 |
| 8 mers | 18 | 18 |
| 9 mers | 10 | 10 |
| 10 mers | 7 | 7 |
| 11 mers | 8 | 8 |
| 12 mers | 3 | 3 |
| 13 mers | 3 | 3 |
| 14 mers | 1 | 1 |
| 15 mers | 1 | 1 |
| 16 mers | 2 | 2 |
| 17 mers | — | — |
| 18 mers | 1 | 1 |
| 19 mers | — | — |
| 20 mers | — | — |
| 21 mers | — | — |
| 22 mers | — | — |
| 23 mers | — | — |
| 24 mers | 1 | 1 |
| 25 mers | 1 | 1 |
| 26 mers | 1 | 1 |
| 27 mers | — | — |
| 28 mers | — | — |
| 29 mers | 1 | 1 |
| 106 mers | 1 | 1 |

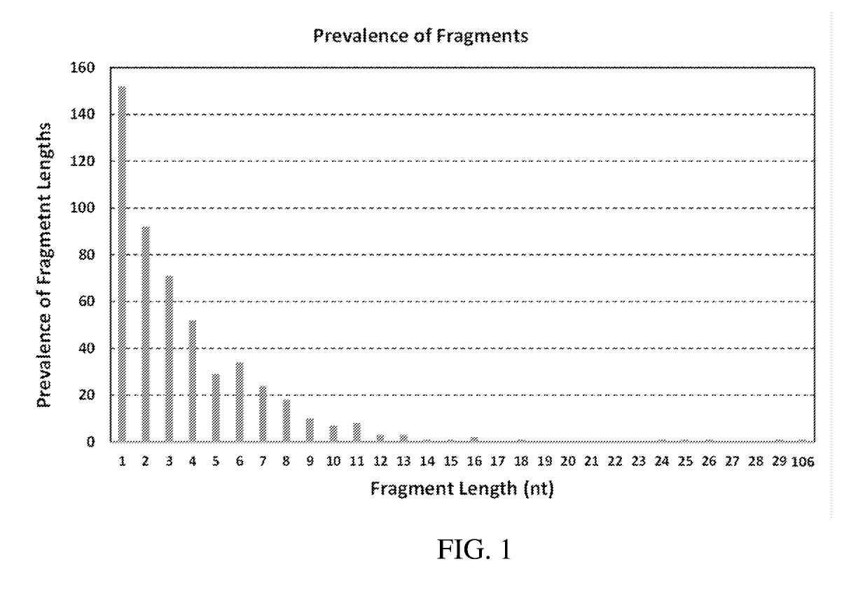

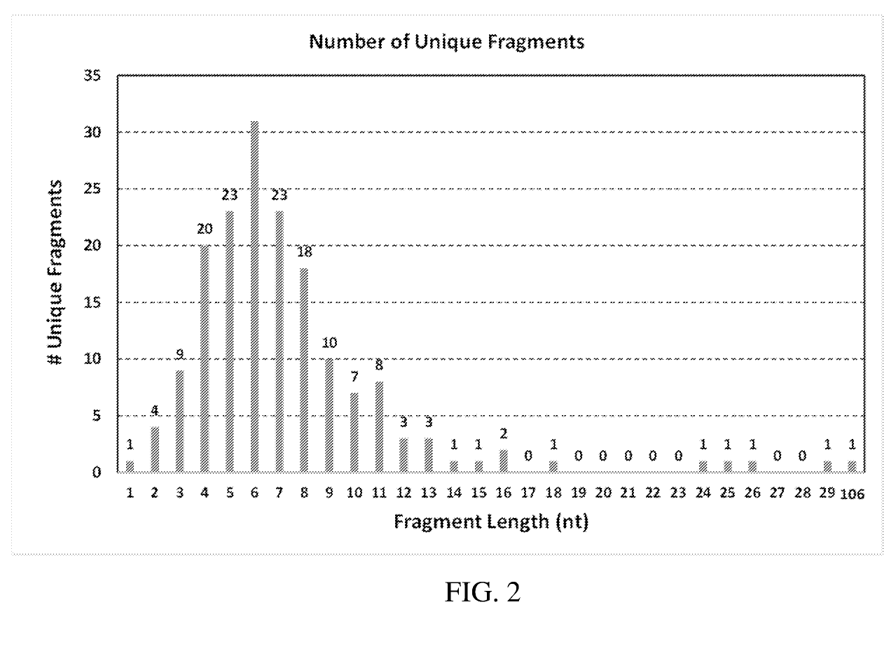

The prevalence of those predicted fragments and the number of unique fragments identified in the mRNA are show in FIGS. 1-2. For example, there are 92 2-mer fragments generated by this digestion as shown in FIG. 1. There are 31 unique 6-mer fragments generated by this RNase digestion, as shown in FIG. 2.

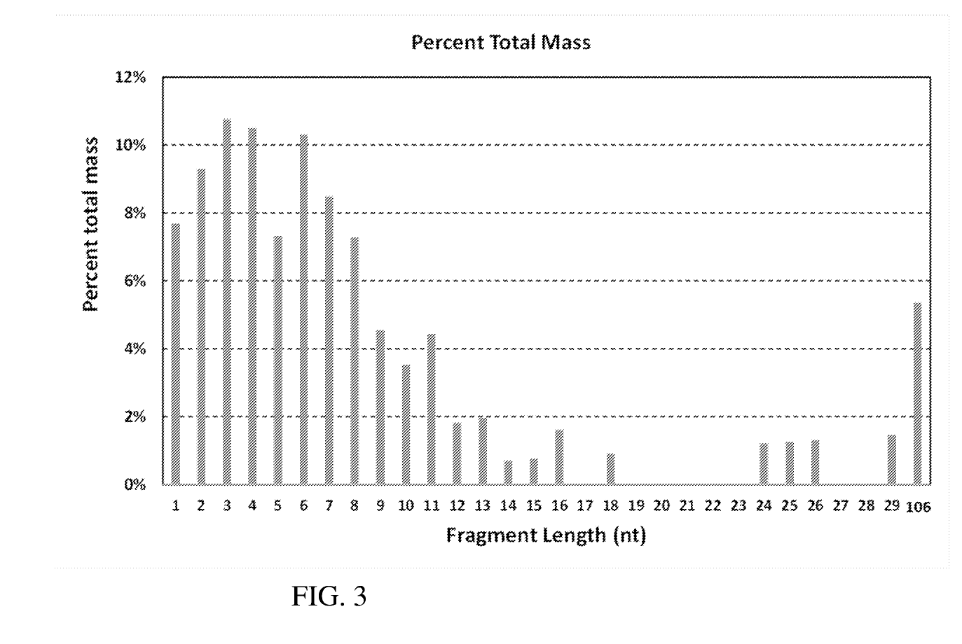

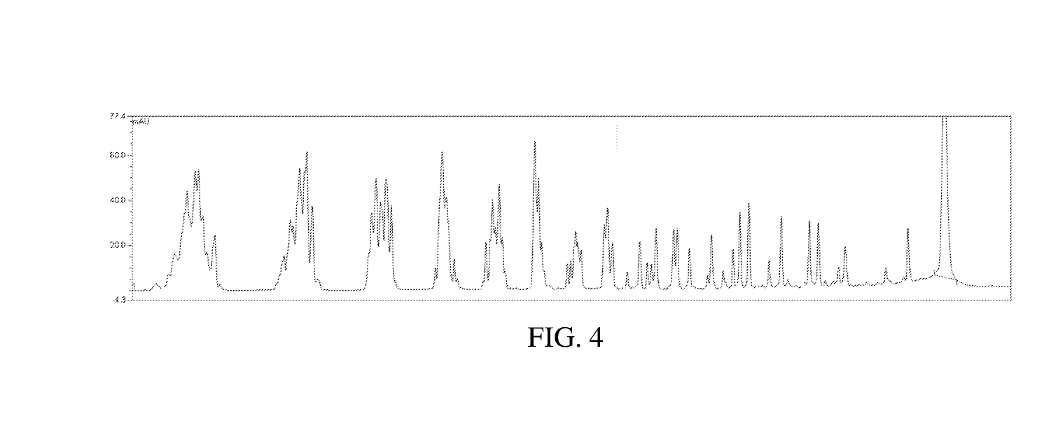

The percent total mass of different fragment lengths is shown in the graph of FIG. 3. For example, 10% of the total mass of the test mRNA sample is digested into 6-mers. FIG. 4 shows analyses of Sample 1 after RNase T1 digestion by HPLC produces a chromatographic pattern that represents a unique fingerprint for Sample 1.

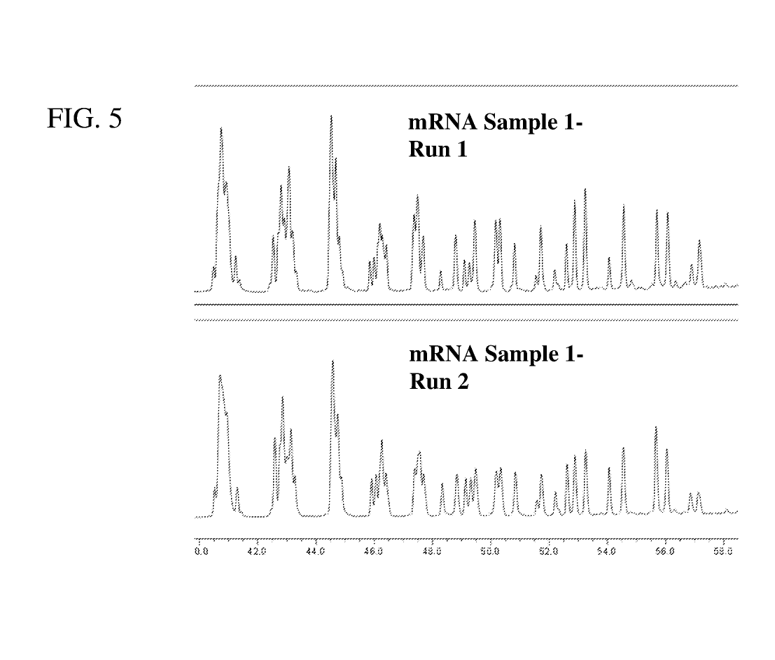

Two test samples of mRNA Sample 1 were digested and run on an HPLC column. FIG. 5 shows representative HPLC data demonstrating the reproducibility of the RNase digestion. The trace patterns for each digestion of mRNA Sample 1 (e.g., Run 1 and Run 2) are almost identical

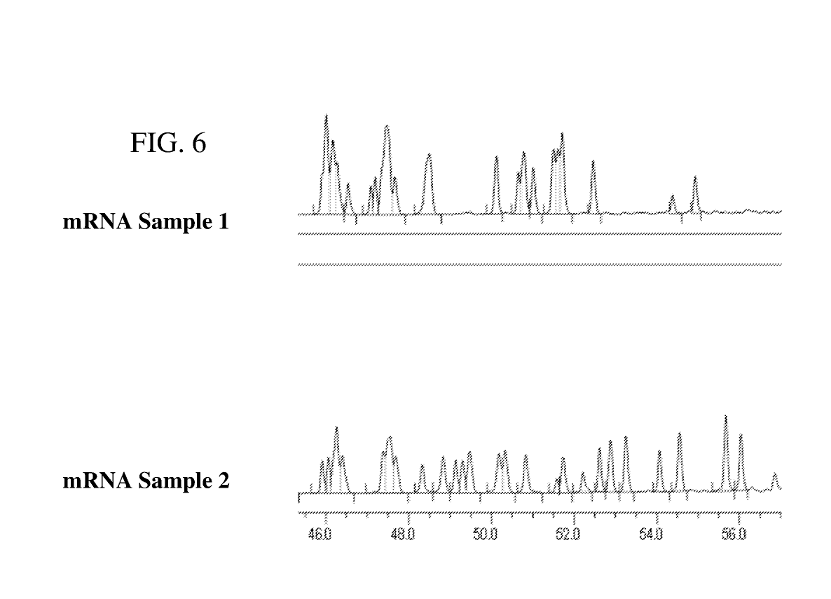

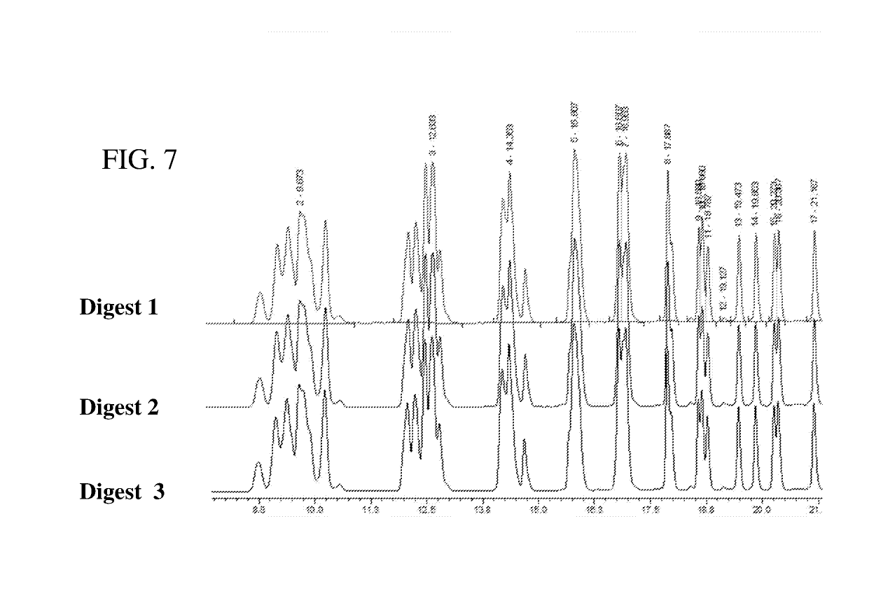

The methods were also performed on different mRNA samples. FIG. 6 shows representative HPLC data demonstrating the unique pattern generated by RNase digestion of two different mRNA samples (e.g., mRNA Sample 1 and mRNA Sample 2). FIG. 7 shows representative HPLC data demonstrating the reproducibility of RNase digestion across multiple digests. Separate aliquots of mRNA Sample 3 were RNase digested (Digest 1, 2 and 3) and run on an HPLC column. The trace patterns for each digestion are almost identical

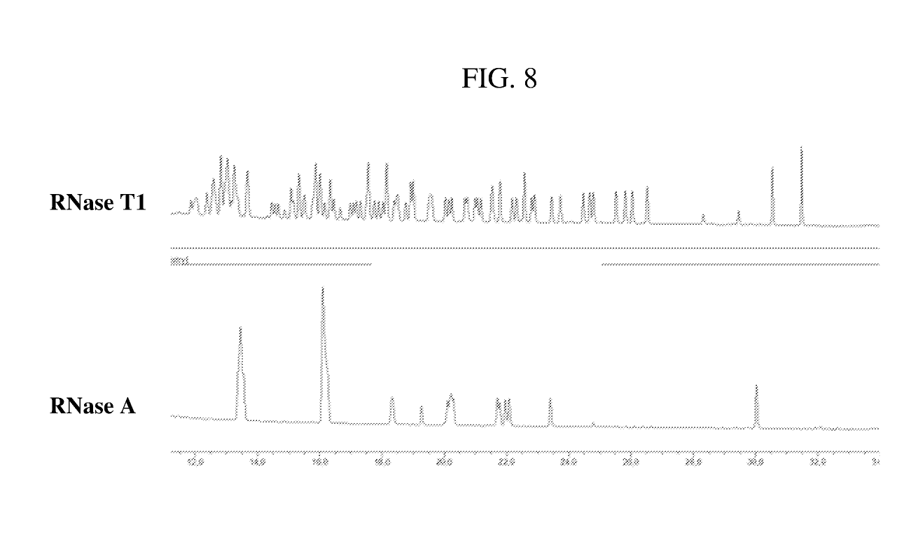

The effect of different RNase enzymes on the analysis methods was also examined. The methods were performed using RNase T1 and RNase A. FIG. 8 shows representative HPLC data illustrating that digestion with different RNase enzymes (e.g., RNase T1 or RNase A) leads to the generation of distinct trace patterns. Digestion of mRNA Sample 3 with RNase T1 provided a more detailed trace pattern than digestion with RNase A.

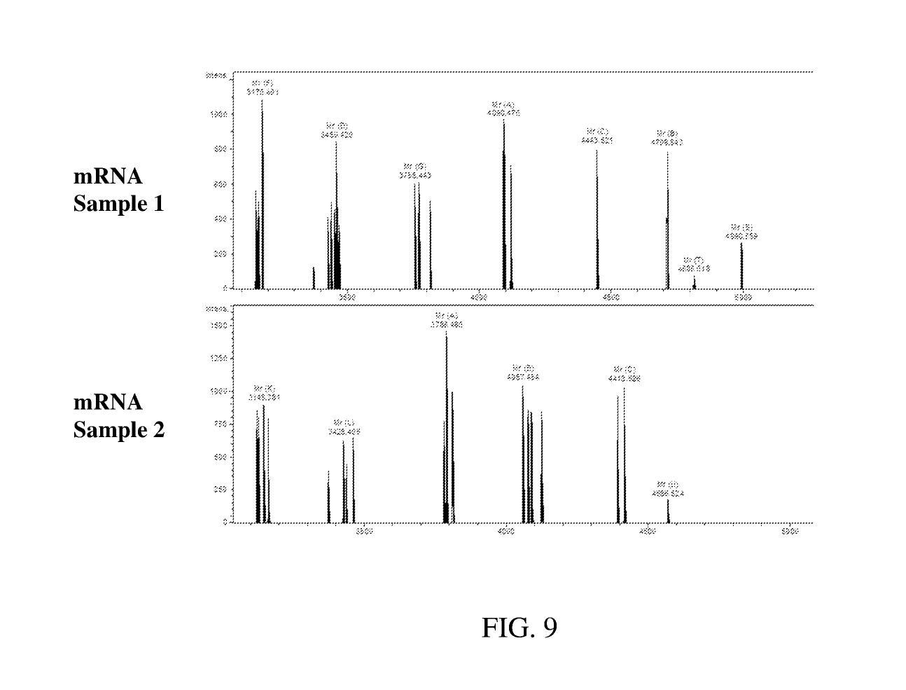

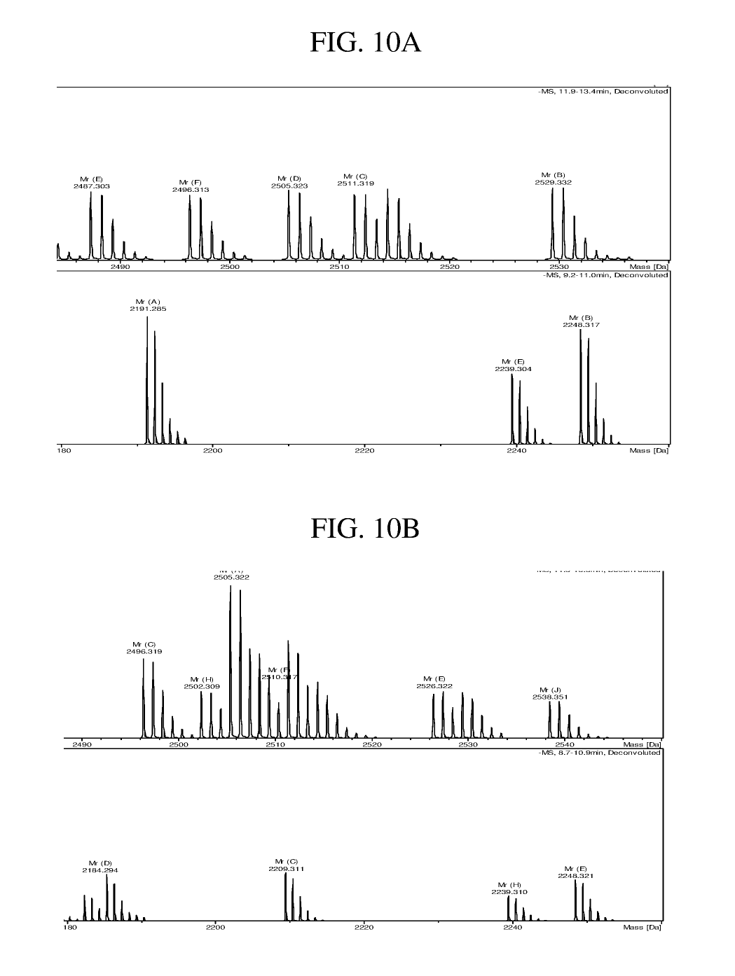

The methods were also performed using different analysis techniques. FIG. 9 shows representative ESI-MS data. Two mRNA samples (mRNA Sample 1 and mRNA Sample 2) were digested with RNase T. ESI-MS was performed on digested samples. Results demonstrated that unique mass traces are generated for each sample. FIGS. 10A-10B show representative data from ESI-MS of two RNase T1-digested mRNA samples (mRNA Sample 4 and mRNA Sample 5). Data demonstrated that each mass fingerprint is unique.

### Example 2: RNase Mapping/Fingerprinting of mCherry mRNA

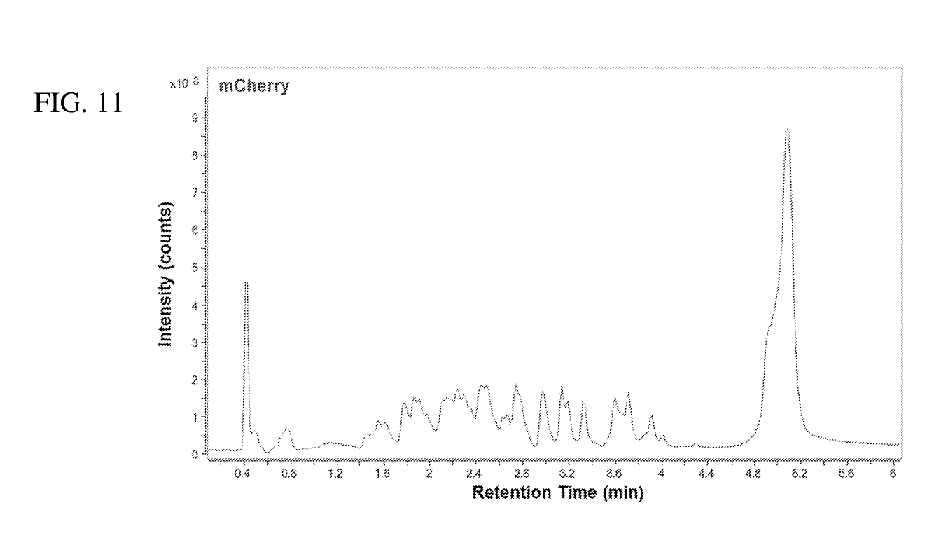

A mRNA sample encoding the fluorescent protein mCherry was processed according the methods described above and LC/MS was performed. Representative data of the LC/MS is shown in FIG. 11.

A total of 43 different oligonucleotide masses were detected. Of these 43 oligos, 28 were unique to a specific location on the mCherry sequence, while 15 were positively identified but could not be localized to a specific location (due to the presence of the same oligo, or isomers thereof, at different locations within the mCherry sequence). Representative data related to the prevalence of digested oligonucleotide fragments and the number of unique fragments identified in the mRNA are show in Table 3. For example, there are 38 2-mer fragments generated by this digestion. There are 5 unique 9-mer fragments generated by this RNase digestion.

TABLE 3
Oligonucleotide fragments produced by RNase T1 digestion of mCherry
mRNA.

| | # Unique | |
|---|---|---|
| | Fragments | Prevalence |
| 2 mers | 0 | 38 |
| 3 mers | 0 | 23 |
| 4 mers | 2 | 2 |
| 5 mers | 4 | 4 |
| 6 mers | 1 | 1 |
| 7 mers | 5 | 5 |
| 8 mers | 5 | 5 |
| 9 mers | 5 | 5 |
| 10 mers | 3 | 3 |
| 12 mers | 2 | 2 |
| 13 mers | 1 | 1 |
| 14 mers | 4 | 4 |
| 16 mers | 2 | 2 |
| 18 mers | 1 | 1 |
| 22 mers | 2 | 2 |
| 24 mers | 1 | 1 |
| 140 mers | 1 | 1 |

Table 4 shows representative data relating to the mass (Da) of the unique fragments identified by RNase T1 digestion of mCherry mRNA.

TABLE 4
Mass of representative mCherry oligonucleotides

| | RET. TIME | | SEQ ID |
|---|---|---|---|
| MASS (Da) | (mins) | SEQUENCES | NO: |
| Unique | | | |
| Sequences | | | |
| 1599.3 | 1.61 | AAAAG | |
| | | UAAG | |
| 2897.49 | 2.78 | AAAUAUAAG | |
| | | AUCAUCAAG | |
| 1579.31 | 1.55 | ACACG | |
| 2209.39 | 2.31 | CCCUAUG | |
| | | ACCACUUCCUUUCG | 1 |
| 1241.24 | 1.28 | CCUG | |
| | | AUAUUCCUG | |
| 2539.43 | 2.43 | ACUAUCUG | |
| | | CUUUCCCG | |
| 2220.38 | 2.31 | AACUUUG | |
| | | UAACCCAAG | |
| 2549.43 | 2.46 | ACAUUAUG | |
| | | ACAUACAAAG | 2 |
| 1928.35 | 2 | AAAAAG | |
| | | UAUAAUG | |
| 2887.49 | 2.85 | AAUAUCAAG | |
| | | AUAUUACUUCACACAAUG | 3 |
| 1589.3 | 1.58 | AACAG | |
| | | UACAAAUG | |
| 2239.38 | 2.23 | AUAAUAG | |
| 1560.3 | 1.5 | CCUCG | |
| | | CUUCUUG | |
| 3829.67 | 3.03 | GCCUCCCCCCAG | 4 |
| | | CCCCUCCUCCCCUUCCUGCACC | 5 |
| | | CG | |
| 2527.47 | 2.31 | UACCCCCG | |
| 46346.1 | 5.09 | C(A₁₄₀) | 6 |

The combined length of all unique oligos was 373 nt, out of a total mRNA length of 1014 nt. Thus, the sequence coverage of the mCherry mRNA by unique oligos was 373/1014=36.8%. When non-unique oligos were considered as well, the sequence coverage jumped to anywhere from 43.9% to 63.8%, depending on whether each identified non-unique oligo originated from just one possible location, or all of the possible locations combined.

Table 5 shows a representative example of a liquid chromatography gradient to obtain preferred separation of components. “A” and “B” are defined as water and acetonitrile, respectively.

TABLE 5
Example Liquid Chromatography (LC) gradient

| Time | A | | Flow | Max Pressure Limit |
|---|---|---|---|---|
| [min] | [%] | B [%] | [mL/min] | [bar] |
| 0.00 | 97.00 | 3.00 | 0.600 | 1200.00 |
| 5.00 | 97.00 | 3.00 | — | — |
| 5.01 | 97.00 | 3.00 | — | — |
| 20.00 | 85.00 | 15.00 | — | — |
| 23.00 | 75.00 | 25.00 | — | — |
| 23.01 | 97.00 | 3.00 | — | — |
| 25.00 | 97.00 | 3.00 | — | — |
| 27.00 | 5.00 | 95.00 | — | — |
| 34.00 | 5.00 | 95.00 | — | — |
| 34.01 | 97.00 | 3.00 | — | — |

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method for determining the presence of an RNA in a mRNA sample, comprising:
determining a signature profile of the mRNA sample, comparing the signature profile to a known signature profile for a test mRNA, identifying the presence of an RNA in the mRNA sample based on a comparison with the known signature profile for the test mRNA.

2. The method of claim 1, wherein the RNA is an impurity in the mRNA sample if the signature profile of the mRNA sample does not match the known signature profile for the test mRNA.

3. The method of claim 2, wherein the method has a sensitivity threshold such that an impurity of less than 1% of the sample is detected.

4. The method of claim 1, further comprising identifying the presence of the test mRNA if the known signature profile for the test mRNA is included within the signature profile of the mRNA sample.

5. The method of any one of claims 1-4, wherein the signature profile of the mRNA sample is determined by a method that includes a digestion step and a separation/detection step.

6. The method of claim 5, wherein the separation/detection step is achieved by a method selected from the group consisting of: gel electrophoresis, capillary electrophoresis, high pressure liquid chromatography (HPLC), and mass spectrometry.

7. The method of claim 6, wherein the HPLC is HPLC-UV.

8. The method of claim 6, wherein the mass spectrometry is Electrospray Ionization mass spectrometry (ESI-MS) or Matrix-assisted Laser Desorption/Ionization-Time of Flight (MALDI-TOF) mass spectrometry.

9. The method of claim 5, wherein the digestion step is a digestion of the mRNA sample with an RNase enzyme to produce a plurality of mRNA fragments.

10. The method of claim 9, wherein the RNase enzyme is RNase T1.

11. The method of claim 10, wherein the RNase T1 is free of glycerol.

12. The method of claim 9, wherein the mRNA sample is mixed with a buffer comprising at least one component selected from the group consisting of: urea, EDTA, magnesium chloride (MgCl₂) and Tris prior to the digestion.

13. The method of claim 12, wherein the mRNA sample and the buffer are incubated at a high temperature to denature the RNA.

14. The method of claim 13, wherein the incubation occurs at about 90° C.

15. The method of claim 9, further comprising incubating the mRNA sample with 2′,3′-Cyclic-nucleotide 3′-phosphodiesterase (CNP) following the digestion to produce a CNP treated mRNA sample.

16. The method of claim 9, wherein the incubating of the mRNA sample with 2′,3′-Cyclic-nucleotide 3′-phosphodiesterase (CNP) is performed for about 1 hour.

17. The method of claim 15, further comprising incubating the CNP treated mRNA sample with Calf Intestinal Alkaline Phosphatase (CIP).

18. The method of any one of claims 15-17, further comprising incubating the mRNA sample with an enzymatic inhibitor to stop the enzyme activity.

19. The method of claim 18, wherein the enzymatic inhibitor is EDTA.

20. The method of claim 19, further comprising incubating the mRNA sample with TEAAc.

21. The method of any one of claims 1-4, wherein the signature profile of the mRNA sample is determined by a method comprising:
digesting the test mRNA with an RNase enzyme to produce a plurality of mRNA fragments;
physically separating the plurality of mRNA fragments;
assigning the signature profile of the mRNA sample by detecting the plurality of fragments;
identifying the presence or absence of the test mRNA by comparing the signature profile of the mRNA sample to the known mRNA signature profile, and
confirming the presence or absence of the test mRNA if the signature profile of the mRNA sample shares identity with the known mRNA signature profile.

22. The method of any one of claims 1-21, wherein the mRNA sample is a sample prepared by an in vitro transcription (IVT) method.

23. The method of any one of claims 1 and 4-22, wherein the RNA is a therapeutic mRNA.

24. The method of any one of claims 1-23, wherein the signature profile of the mRNA sample is in the form of an absorbance spectrum or a mass spectrum.

25. The method of any one of claims 1-23, wherein the signature profile of the mRNA sample shares at least 70%, at least 80%, at least 90%, at least 95%, at least 99%, or at least 99.9% identity with the known signature profile for the test mRNA.

26. The method of claim 2, wherein the RNA that is identified as an impurity is removed from the mRNA sample using a separation step to produce a pure product.

27. A pure mRNA sample, comprising: a composition of an in vitro transcribed (IVT) RNA and a pharmaceutically acceptable carrier, wherein the composition is prepared according to the method of claim 26.

28. A method for quality control of an RNA pharmaceutical composition, comprising
digesting the RNA pharmaceutical composition with an RNase enzyme to produce a plurality of RNA fragments;
physically separating the plurality of RNA fragments;
generating a signature profile of the RNA pharmaceutical composition by detecting the plurality of fragments;
comparing the signature profile with a known RNA signature profile, and
determining the quality of the RNA based on the comparison of the signature profile with the known RNA signature profile.

29. The method of claim 28, wherein an impurity is detected in the RNA pharmaceutical composition if the signature profile of the RNA pharmaceutical composition does not match the known RNA signature profile.

30. The method of claim 28, wherein the separating step is achieved by a method selected from the group consisting of: gel electrophoresis, capillary electrophoresis, high pressure liquid chromatography (HPLC), and mass spectrometry.

31. The method of claim 30, wherein the HPLC is HPLC-UV.

32. The method of claim 30, wherein the mass spectrometry is Electrospray Ionization mass spectrometry (ESI-MS) or Matrix-assisted Laser Desorption/Ionization-Time of Flight (MALDI-TOF) mass spectrometry.

33. The method of claim 32, further comprising incubating the RNA pharmaceutical composition with 2′,3′-Cyclic-nucleotide 3′-phosphodiesterase (CNP) following the digestion to produce a CNP treated RNA pharmaceutical composition.

34. The method of claim 33, wherein the incubating of the RNA pharmaceutical composition with 2′,3′-Cyclic-nucleotide 3′-phosphodiesterase (CNP) is performed for about 1 hour.

35. The method of claim 33, further comprising incubating the CNP treated RNA pharmaceutical composition with Calf Intestinal Alkaline Phosphatase (CIP).

36. The method of any one of claims 33-35, further comprising incubating the RNA pharmaceutical composition with an enzymatic inhibitor to stop the enzyme activity.

37. The method of claim 36, wherein the enzymatic inhibitor is EDTA.

38. A system for determining batch purity of an RNA pharmaceutical composition comprising: a computing system; at least one electronic database coupled to the computing system; at least one software routine executing on the computing system which is programmed to: (a) receive data comprising an RNA fingerprint of the RNA pharmaceutical composition; (b) analyze the data; (c) based on the analyzed data, determine batch purity of the RNA pharmaceutical composition.

39. A method for determining the presence of an RNA in a mRNA sample, comprising:
determining a signature profile of the mRNA sample, comparing the signature profile to a theoretical mass pattern comprising predicted masses of fragments from the primary molecular sequence of the mRNA, identifying the presence of an RNA in the mRNA sample based on the theoretical versus observed mass pattern.

40. The method of claim 39, wherein the RNA is an impurity in the mRNA sample if the signature profile of the mRNA sample does not match the theoretical mass pattern.

41. The method of claim 40, wherein the method has a sensitivity threshold such that an impurity of less than 1% of the sample is detected.

42. The method of claim 39, further comprising identifying the presence of the test mRNA if the theoretical mass pattern for the test mRNA is included within the signature profile of the mRNA sample.

43. The method of any one of claims 39-42, wherein the signature profile of the mRNA sample is determined by a method that includes a digestion step and a separation/detection step.

44. The method of claim 43, wherein the separation/detection step is achieved by a method selected from the group consisting of: gel electrophoresis, capillary electrophoresis, high pressure liquid chromatography (HPLC), and mass spectrometry.

45. The method of claim 44, wherein the HPLC is HPLC-UV.

46. The method of claim 44, wherein the mass spectrometry is Electrospray Ionization mass spectrometry (ESI-MS) or Matrix-assisted Laser Desorption/Ionization-Time of Flight (MALDI-TOF) mass spectrometry.

47. The method of claim 43, wherein the digestion step is a digestion of the mRNA sample with an RNase enzyme to produce a plurality of mRNA fragments.

48. The method of claim 47, wherein the RNase enzyme is RNase T1.

49. The method of claim 48, wherein the RNase T1 is free of glycerol.

50. The method of claim 47, wherein the mRNA sample is mixed with a buffer comprising at least one component selected from the group consisting of: urea, EDTA, magnesium chloride (MgCl₂) and Tris prior to the digestion.

51. The method of claim 50, wherein the mRNA sample and the buffer are incubated at a high temperature to denature the RNA.

52. The method of claim 51, wherein the incubation occurs at about 90° C.

53. The method of claim 47, further comprising incubating the mRNA sample with 2′,3′-Cyclic-nucleotide 3′-phosphodiesterase (CNP) following the digestion to produce a CNP treated mRNA sample.

54. The method of claim 47, wherein the incubating of the mRNA sample with 2′,3′-Cyclic-nucleotide 3′-phosphodiesterase (CNP) is performed for about 1 hour.

55. The method of claim 53, further comprising incubating the CNP treated mRNA sample with Calf Intestinal Alkaline Phosphatase (CIP).

56. The method of any one of claims 53-55, further comprising incubating the mRNA sample with an enzymatic inhibitor to stop the enzyme activity.

57. The method of claim 56, wherein the enzymatic inhibitor is EDTA.

58. The method of claim 57, further comprising incubating the mRNA sample with TEAAc.

59. The method of any one of claims 39-42, wherein the signature profile of the mRNA sample is determined by a method comprising:
digesting the test mRNA with an RNase enzyme to produce a plurality of mRNA fragments;
physically separating the plurality of mRNA fragments;
assigning the signature profile of the mRNA sample by detecting the plurality of fragments;
identifying the presence or absence of the test mRNA by comparing the signature profile of the mRNA sample to the theoretical mass pattern, and
confirming the presence or absence of the test mRNA if the signature profile of the mRNA sample shares identity with the theoretical mass pattern.

60. The method of any one of claims 39-59, wherein the mRNA sample is a sample prepared by an in vitro transcription (IVT) method.

61. The method of any one of claims 39 and 42-60, wherein the RNA is a therapeutic mRNA.

62. The method of any one of claims 39-61, wherein the signature profile of the mRNA sample is in the form of an absorbance spectrum or a mass spectrum.

63. The method of any one of claims 39-61, wherein the signature profile of the mRNA sample shares at least 70%, at least 80%, at least 90%, at least 95%, at least 99%, or at least 99.9% identity with the theoretical mass pattern.

64. The method of claim 40, wherein the RNA that is identified as an impurity is removed from the mRNA sample using a separation step to produce a pure product.

65. A pure mRNA sample, comprising: a composition of an in vitro transcribed (IVT) RNA and a pharmaceutically acceptable carrier, wherein the composition is prepared according to the method of claim 64.

66. A method for quality control of an RNA pharmaceutical composition, comprising
digesting the RNA pharmaceutical composition with an RNase enzyme to produce a plurality of RNA fragments;
physically separating the plurality of RNA fragments;
generating a signature profile of the RNA pharmaceutical composition by detecting the plurality of fragments;
comparing the signature profile with a theoretical mass pattern comprising predicted masses of fragments from the primary molecular sequence of the mRNA, and
determining the quality of the RNA based on the comparison of the signature profile with the theoretical mass pattern.

67. The method of claim 66, wherein an impurity is detected in the RNA pharmaceutical composition if the signature profile of the RNA pharmaceutical composition does not match the theoretical mass pattern.

68. The method of claim 66, wherein the separating step is achieved by a method selected from the group consisting of: gel electrophoresis, capillary electrophoresis, high pressure liquid chromatography (HPLC), and mass spectrometry.

69. The method of claim 68, wherein the HPLC is HPLC-UV.

70. The method of claim 68, wherein the mass spectrometry is Electrospray Ionization mass spectrometry (ESI-MS) or Matrix-assisted Laser Desorption/Ionization-Time of Flight (MALDI-TOF) mass spectrometry.

71. The method of claim 70, further comprising incubating the RNA pharmaceutical composition with 2′,3′-Cyclic-nucleotide 3′-phosphodiesterase (CNP) following the digestion to produce a CNP treated RNA pharmaceutical composition.

72. The method of claim 71, wherein the incubating of the RNA pharmaceutical composition with 2′,3′-Cyclic-nucleotide 3′-phosphodiesterase (CNP) is performed for about 1 hour.

73. The method of claim 71, further comprising incubating the CNP treated RNA pharmaceutical composition with Calf Intestinal Alkaline Phosphatase (CIP).

74. The method of any one of claims 71-73, further comprising incubating the RNA pharmaceutical composition with an enzymatic inhibitor to stop the enzyme activity.

75. The method of claim 74, wherein the enzymatic inhibitor is EDTA.
